(19) United States
(12) Patent Application Publication
(10) Pub. No.: US 2008/0219405 A1
(43) Pub. Date: Sep. 11, 2008

(54) System and method for patient setup for radiotherapy treatment

(76) Inventors: Tony Falco, La Prairie (CA); Martin Lachaine, St. Laurent (CA); Martin Bonneville, Montreal (CA)

Correspondence Address: GOODWIN PROCTER LLP;PATENT ADMINISTRATOR, EXCHANGE PLACE, BOSTON, MA, 02109-2881, US

(21) Appl. No.: 11/516,722

(22) Filed: Sep. 6, 2006

Related U.S. Application Data
(60) Provisional application No. 60/714,397, filed on Sep. 6, 2005.

(51) Int. Cl. A61N 5/10

(52) U.S. Cl. 378/65

(57) ABSTRACT

Positioning an anatomical feature of a patient during repeated radiotherapy treatments, and accounting for variations in that position between treatments allow a patient to be placed in a substantially repeatable orientation with respect to a treatment device.

### CROSS-REFERENCE TO RELATED APPLICATIONS

This application claims priority to and the benefit of U.S. provisional patent application Ser. No. 60/714,397, filed Sep. 6, 2005, the entire disclosure of which is hereby incorporated herein by reference in its entirety.

### FIELD OF THE INVENTION

The present invention relates generally to the field of radiotherapy, and more particularly to positioning an anatomical feature of a patient during repeated treatments, and accounting for variations in positioning between and/or during treatments.

### BACKGROUND OF THE INVENTION

Cancerous tumors on or within an anatomical feature of a patient are often treated using radiation therapy involving one or more radiation-emitting devices. The primary goal of radiation therapy is the complete eradication of the cancerous cells, while the secondary goal is to avoid, to the maximum possible extent, damaging healthy tissue and organs in the vicinity of the tumor. Typically, a radiation therapy device includes a gantry that can be rotated around a horizontal axis of rotation during the delivery of a therapeutic treatment. A particle linear accelerator (“LINAC”) is located within the gantry, and generates a high-energy radiation beam of therapy, such as an electron beam or photon (x-ray) beam. The patient is placed on a movable treatment table located near the isocenter of the gantry, and the radiation beam is directed towards the tumor or lesion to be treated.

Radiation therapy typically involves a planning stage and a treatment stage. In the planning stage, an X-ray computed tomography (CT) scanner (or similar device) is used to acquire images of a lesion. These images are used to accurately measure the location, size, contour, and number of lesions to be treated, in order to establish an isocenter, a dose distribution, and various irradiation parameters. These parameters are then used to prepare a treatment plan designed to irradiate the lesion while minimizing damage to the surrounding healthy tissue.

The treatment plan designed during the treatment planning session is then used in delivering radiation during one or more treatment delivery sessions. Generally, treatment delivery occurs within a few days or weeks of the preparation of the treatment plan, and can include one or more sessions, depending on the type of lesion being treated, the radiosensivity of surrounding healthy organs, as well as other factors.

A significant problem with the preparation of a treatment plan and the ensuing treatment delivery is that the lesion or lesions being treated and the tissue and organs surrounding the lesion can undergo morphological changes and shifts between the planning stage and treatment delivery, as well as between each treatment session. As a result, the radiation called for in the treatment plan may not be delivered in the proper location and/or at the dosage required when treatment is actually carried out. In some instances, the treatment delivery sessions can occur over a period of weeks or even months, giving rise to further uncertainties in patient positioning and physiology. Other factors such as, but not limited to, sagging of external anatomy, weight change of the patient, muscular changes (through wastage, injury, or exercise) may also result in changes to the anatomical structure of the lesion and surrounding tissue and organs from one treatment session to the next.

Whole-breast radiotherapy, for example, involves uniformly treating the entire affected breast, including the chest wall, while attempting to minimize any dose that may affect the lung. Typically, this is accomplished with a set of opposing “tangent” beams which are designed on a CT planning image acquired prior to a first treatment session. Depending on the stage of the cancer, beams may be added to treat nodes, such as the supraclavicular nodes. These extra beams must be carefully matched to the tangent beams to avoid overlap, which would result in regions of excessive dose. The beams are designed during simulation and treatment planning stages, which involves selection of field size (i.e., beam aperture), isocenter placement (of the beams relative to the patient), selection of wedges (which preferentially attenuate parts of the beam), and beam weights (how much radiation is delivered from each beam) such that the prescribed dose is delivered across the breast. Other forms of delivery exist, such as Intensity-Modulated Radiation Therapy (IMRT), which modulates the beam intensities to achieve a more uniform dose distribution. Dose distributions for a particular beam arrangement are calculated by a treatment planning computer and approved by the physician.

Once a treatment plan is designed, the patient is placed on the treatment couch (hopefully in the same position assumed during the CT scan) for each of the treatment sessions, and the treatment is executed according to the treatment plan. Patient positioning devices such as breast boards are often used to ensure consistency of the patient's position across treatment sessions. The patient can be treated with one arm raised and held in place with an arm holder, for example, giving the lateral beam direct access to the breast to be treated. External marks placed on the patient's skin at the time of the CT scan (usually tattoos) may also be used to place the patient correctly relative to orthogonal sighting lasers affixed in the treatment room. Despite these aids in treatment setup, studies have shown that it is difficult to place the patient in the same manner for treatment planning and each treatment delivery session such that the radiation dosage is delivered accurately. For example, the patient may be rotated, and/or the breast can be deformed or displaced relative to the original CT. This compromises the delivery of the dose distribution intended by the treatment plan.

To circumvent these issues, it has been proposed to use a camera system installed in the treatment room to obtain external surface information from the patient, and, based on images obtained from the cameras. While this approach may be able to compensate for changes in the patient's external surface, changes in internal anatomy (which can occur on a daily basis) are not considered. For example, the lung/chest wall interface position relative to the patient surface can change daily, especially if the patient's arm position is not reproducible. This interface is important since the whole breast, including the chest wall, must be treated uniformly while maintaining a minimal amount of radiation dose to the lung and/or heart.

It has also been proposed to incorporate a CT, cone-beam CT, MRI or other tomographic imager in the treatment room itself. The internal anatomy and external surface can thereby be visualized, and potentially the treatment parameters (e.g., isocenter placement, beam angles, etc.) can be modified to compensate for daily changes in patient setup. This approach, however, is expensive, bulky, and subjects the patient to additional radiation.

As a result, a convenient and harmless approach is needed to detect changes in patient positioning based on both surface and internal shifts of the patient's anatomy.

### SUMMARY OF THE INVENTION

The invention incorporates information obtained from the surface of a patient's anatomy with images of the patient's internal anatomy (such as, in the case of breast treatment, the lung/chest wall interface) during radiotherapy planning and treatment to correct for patient setup errors and/or changes to anatomical characteristics. An image or model of the patient's external surface in the general area of the lesion is obtained prior to treatment using, for example, a camera system or a physical digitizing pointer tool. Surface information can include both natural and/or artificial markings such as tattoos and delineations of field outline. For example, an image of the chest wall, pleura and/or lung surface may be obtained using two-dimensional or three-dimensional ultrasound imaging techniques. The surface information and ultrasound information, although acquired with different devices, are referenced in the same coordinate system through proper calibration of the imaging devices relative to the patient and/or the room. Radiotherapy treatment parameters, such as an isocenter, a couch angle, a beam angle, a radiation dosage, a wedge angle, a collimator size, a collimator shape, and/or a collimator angle are modified or adapted to account for the actual breast, lung, and chest wall positions and shapes determined just prior to treatment delivery, which are more accurate than those obtained at the time of planning. These treatment parameters govern the treatment dose and how and where it is delivered to the patient.

For deep internal organs that may require radiation treatment, such as the prostate, slight differences in the location of the region of interest within the patient from one treatment session to another can be corrected for by simply shifting the treatment couch to realign the region to its planning position. Differences and shifts in the external anatomy are of secondary importance and may have minimal effect on the required treatment plan. This is due, at least in part, to the fact that slight differences in the depth, and thus attenuation, of the radiation beam through the body are less significant when the depths are large. As a result, slight differences in the distance from the surface of the skin to the treatment region do not have a great impact on the radiation dose delivered to that region. In the treatment of deeply located organs, therefore, the value of obtaining both internal anatomical information and external information prior to every treatment session is limited. A simple repositioning of the patient may be made to compensate for anatomical changes when treating deeply located lesions.

For cancerous tissue located near the surface of the skin, however, such as lesions within a patient's breast, attenuation of a radiation beam passing through this region can produce a significant change in the radiation actually received at the lesion. As a result, it is very important when treating near surface lesions to know both the location of the treatment region and the depth of this region below the surface of the skin. The present invention, by using both external information (in order to correctly locate the treatment region with respect to the patient) and internal anatomical information (to correctly measure the depth of that region below the surface), accurately corrects for morphological and conformational changes to provide the desired dose to the proper anatomical region. Thus, the approach of the present invention is especially useful when treating near-surface lesions, or lesions encompassed within a surface which can deform significantly. By contrast, prior techniques for locating breast lesions for treatment, which generally align the breast using previously created external markings alone, do not account for possible changes in the depth of the lesion below the surface of the skin.

The invention is particularly useful in connection with imaging modalities, such as ultrasound, that do not themselves provide surface information. But it is equally applicable wherever three-dimensional surface information is not conveniently obtainable from internal images. For example, some nuclear medicine imaging modalities, such as PET or SPECT, tend to show strong signals where there is uptake (e.g., at tumor sites) but weak signals elsewhere (e.g., at the skin surface). Indeed, even though conventional CT techniques reveal surface information, that information must usually be extracted using, for example, a threshold algorithm that may be inconvenient or inaccurate. Finally, if fiducials are implanted inside a tumor, conventional projection x-rays will not provide three-dimensional surface information. This can occur, for instance, when a surgeon removes a tumor but leaves surgical clips around the tumor bed. These can be detected with a set of two or more projection x-ray images which will characterize the internal anatomy and suggest how it should be placed relative to a treatment beam, but surface information cannot readily be extracted from these projection images.

In one exemplary embodiment, both external information and internal anatomical information are gathered and stored at the time of creation of a treatment plan. This may include, but is not limited to, producing an external map of a breast (and placing marks on a patient's skin to identify set locations on that external map), and producing an internal anatomical map of the breast to identify both the depth of the lesion (or lesions) below the surface of the skin and the location of other anatomical features (such as, but not limited to, the pleura, the ribs, and the lungs) with respect to the lesion(s). This information is then used by a medical practitioner to create a treatment plan for the breast, allowing the lesion(s) to be treated with the appropriate radiation dose while limiting the radiation delivered to the surrounding healthy tissue and/or organs.

At the time of each required treatment, the internal and external anatomical measurements are repeated. The positions of the markings on the skin, and the positions and depth of the lesion(s) and surrounding anatomical features, can then be compared to the information taken during the creation of the treatment plan. If changes in the external and/or internal anatomical position information are found, the location of the patient and/or the treatment plan can be changed to compensate for this anatomical change, and to ensure that the required treatment dose is delivered to the proper location.

It should be noted that it is often desirable to treat cancerous tissue in a patient's breast by delivering a uniform dose to the entire breast, although in an alternative embodiment, it may also be desirable to deliver a more localized dose to a specific region of the breast. In either case, identification of both the external and internal anatomy will be useful to ensure that the correct dosage is delivered, either to the entire breast or the specific portion of the breast, as required. For example, unless accounted for at each treatment session, changes in the shape of the breast over time may result in the previously prepared treatment plan not providing the entire breast with a uniform dosage, or result in part of a breast not receiving any dose.

Accordingly, in a first aspect, the invention provides a method for determining an adjustment to a radiation treatment plan that includes obtaining a radiation treatment plan having various treatment parameters that describe the positioning of a patient to be treated with radiation with respect to external and internal anatomical features of the patient. Further, an image of both an external feature of the patient (using, for example, a camera, a tracking tool, or a laser scanning device) and an image of an internal anatomical feature of the patient (using, for example, a two-dimensional or three-dimensional ultrasound imager or an x-ray imaging device) are obtained, each using a respective reference coordinate system, and taken at substantially the same time. For the purposes of the present invention, “substantially the same time” and “contemporaneously” connote a period of time over which changes in the location of the patient's anatomy are unlikely to occur, such that the surface and internal anatomical information will produce a consistent geometrical data set for the patient's treatment area. This time scale will usually involve a single treatment session, which may encompass a number of minutes or hours.

In general, a visual representation of at least one external feature is used to determine an adjustment required in at least one radiotherapy beam parameter (e.g., the beam angle, collimator shape, etc.), while the visual representation of at least one internal anatomical feature is typically used to determine an adjustment required in at least one patient-position parameter (e.g., the couch angle or couch position). But a sufficiently large change in the visual representation may indicate the need for adjustment of both the beam and the patient, e.g., if a bodily deformation is simply too great to be accommodated by changes in the beam; and similarly, a sufficiently large internal change may indicate the need to adjust the beam, e.g., if the tumor to be treated has not only shifted but grown. Moreover, a threshold value may be set, below which an adjustment of one or more treatment parameters is not required, and a threshold value may also be set above which a full recalculation of the treatment plan is required.

One or more of the treatment parameters are then adjusted to compensate for changes in the patient's position relative to a radiation treatment device based on the internal anatomical feature of the patient and the external feature representation. The visual representation obtained using an ultrasound imaging device may produce a two-dimensional image and then maps the two-dimensional image into three-dimensional space.

The external feature can be a naturally occurring feature (such as a freckle, or in the case of breast treatment, the areola) or an artificial feature such as a tattoo or ink mark placed on the patient's skin for reference. The treatment parameters can include the isocenter of the radiation treatment device, a beam angle, a couch angle, a couch position, a radiation dosage, a wedge angle, a collimator size, a collimator shape and/or a collimator angle. In some embodiments, the two reference coordinate systems are the same coordinate system, whereas in other embodiments they are related to each other through a transformation (e.g. an affine transformation).

In another aspect, a system for determining an adjustment to a radiation treatment plan includes a receiver for receiving a radiation treatment plan, a visual representation of a patient's external feature and a visual representation of a patient's internal anatomical feature, and a treatment positioning module. The radiation treatment plan includes various treatment parameters that describe the location of a patient with respect to the external features and internal anatomical features. The visual representation of the patient's external feature is referenced to a first reference coordinate system, and the visual representation of the patient's internal feature is referenced to a second reference coordinate system. Based on the radiation treatment plan and the received visual representations, the treatment positioning module adjusts one or more of the treatment parameters to compensate for changes in the position of the patient with respect to their internal anatomy.

In some embodiments, the system further includes a camera for obtaining the visual representation of the patient's external feature. The system can also include an ultrasound imaging device for obtaining the visual representation of the patient's internal anatomy, and can further include an optical tracking device for monitoring the location of the ultrasound device with respect to the second reference coordinate system.

In another aspect, a method for determining a radiation treatment plan includes obtaining a visual representation of an external feature of a patient in reference to a reference coordinate system and at substantially the same time as the external-feature visual representation is obtained, obtaining a visual representation of an internal anatomical feature of the patient in reference the reference coordinate system. Further, the method includes determining a radiation treatment plan (including the relevant treatment parameters) relative to the reference coordinate system based on the position of the patient relative to the external and internal anatomical features of the patient.

The radiation treatment plan may be determined at substantially the same time as the visual representation of the internal anatomical feature of the patient is obtained, as well as at substantially the same time as the radiation treatment is delivered to the patient.

### BRIEF DESCRIPTION OF THE DRAWINGS

The objects and features of the invention can be better understood with reference to the drawings described below, and the claims. The drawings are not necessarily to scale, emphasis instead generally being placed upon illustrating the principles of the invention. In the drawings, like numerals are used to indicate like parts throughout the various views.

FIG. 1A is a schematic view of the chest region of a patient;

FIG. 1B is a schematic cross-section of a breast and associated coordinate system in accordance with one embodiment of the invention;

FIG. 2A is schematically illustrates of a pointer tool based position measurement system for the chest of a patient in accordance with one embodiment of the invention;

FIG. 2B is a schematic view of a camera-based position-measurement system and associated coordinate system for the chest region of a patient in accordance with one embodiment of the invention; and

FIG. 3 is a schematic cross-section of a internal anatomical imaging system imaging a patient's breast in accordance with one embodiment of the invention;

FIG. 4A is a schematic cross-section of a radiation beam treating a patient's breast, and an associated coordinate system, prior to realignment in accordance with one embodiment of the invention;

FIG. 4B is a schematic cross-section of the radiation beam and associated coordinate system of FIG. 4B after realignment in accordance with one embodiment of the invention;

FIG. 5A is a flow chart illustrating one method of positioning a patient for treatment in accordance with one embodiment of the invention;

FIG. 5B is a flow chart illustrating a second method of positioning a patient for treatment in accordance with one embodiment of the invention;

FIG. 5C is a flow chart illustrating a third method of positioning a patient for treatment in accordance with one embodiment of the invention; and

FIG. 6 schematically illustrates a system for determining adjustments to a radiation treatment plan according to an embodiment of the invention.

### DETAILED DESCRIPTION

Throughout the following descriptions and examples, the invention is described in the context of positioning a patient in preparation for the delivery of radiation therapy to a breast. However, it is to be understood that the present invention may be applied in cases in which a patient is positioned in anticipation of receiving any position-based treatment and for any anatomical feature of the body, be it internal (e.g., a tumor within the breast surgical bed) or external (e.g., a melanoma on the skin).

In one embodiment, the invention generally involves four phases: receiving a previously defined treatment plan, obtaining patient surface information, obtaining internal anatomical information, and correcting the treatment plan. In some embodiments, however, the treatment plan can be developed just prior to treatment, even while the patient is in the treatment room awaiting delivery of radiotherapy. Although such an approach minimizes positioning errors between the planning stage and the first treatment, radiation therapy and other forms of treatment often require multiple treatment sessions spaced over a period of days, weeks or months. The methods and systems described herein therefore also address potential positioning errors that arise from one treatment session to the next and/or subsequent treatment sessions.

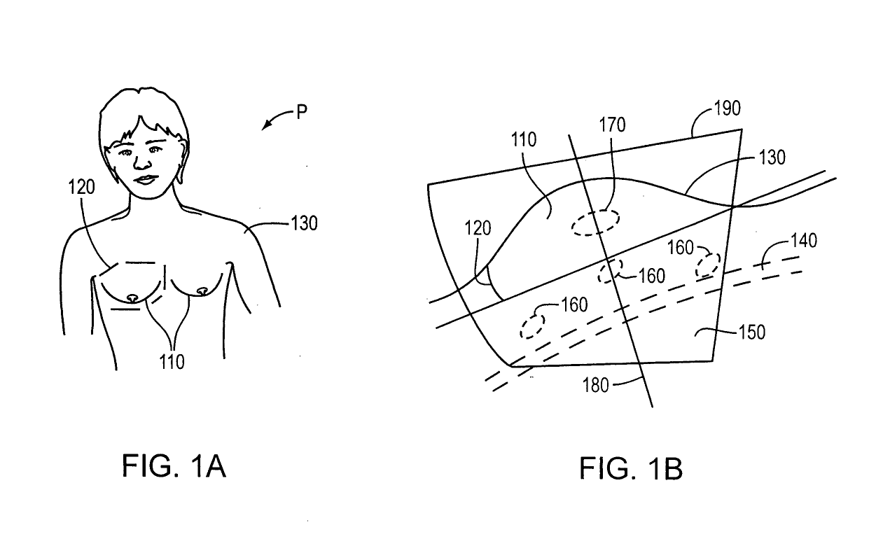

An example chest region of a patient is shown in FIG. 1A in which patient P, having been diagnosed with breast cancer, is treated using radiotherapy techniques to eradicate the cancerous lesion(s) from her breast 110. To facilitate the treatment planning and irradiation of the lesion or lesions, one or more marks 120 are placed about the breast 110 on the patient's skin (indicated generally at 130). These marks 120 can be used to determine, to a first approximation, proper positioning of the patient P during the numerous treatment sessions that may be required. These marks 120 may be permanently or semi-permanently tattooed or painted on the skin 130 to provide positioning information to a medical practitioner from one treatment to the next.

A cross-section of the general anatomical structures of interest when treating a cancerous breast lesion is shown in FIG. 1B. The structures of interest include the patient's skin 130 on which the marks 120 are placed, the chest/lung interface (the pleura) 140, the lung 150, ribs 160, and the lesion 170 that requires treatment. Superimposed on these structures is a coordinate system 180 centered on the determined treatment isocenter of the lesion 170. A cross-section of the resulting radiation beam 190 associated with the coordinate system is also shown.

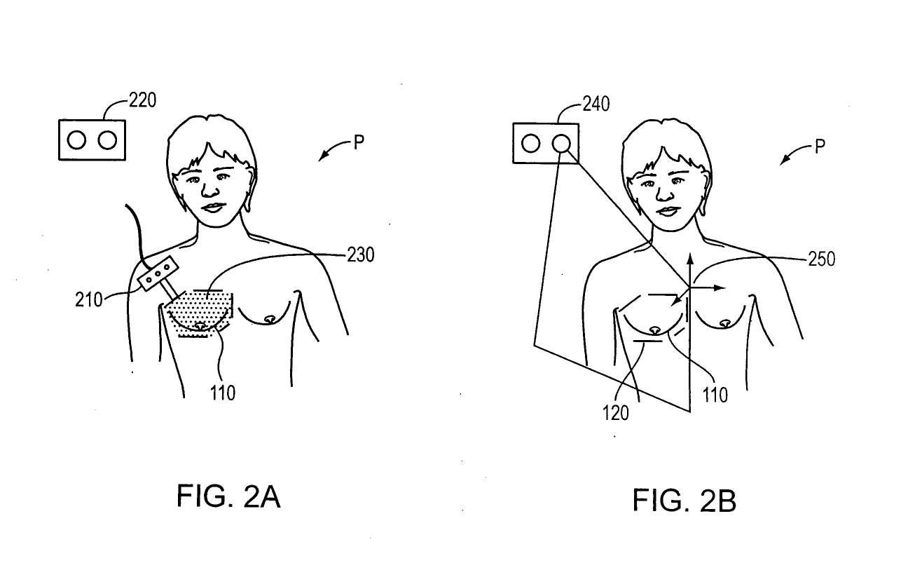

Surface information and/or skin markings within the region of interest on a patient's skin may be acquired in a number of ways. In one embodiment of the invention, a discrete number of locations on the skin of a patient can be measured. An exemplary system for discrete surface measurement is shown in FIG. 2A. In this embodiment, surface information and/or skin markings are acquired in the treatment room on each treatment day while the patient P is in the required treatment position. Surface measurements may be performed using a pointer tool 210 tracked by a tracking system 220, such as, but not limited to, an optical camera, a magnetic camera, or a laser scanning system. To obtain surface measurement information, a user points the tool 210 at a selected number of points 230 on the surface of the patient in the vicinity of the breast 110 to be treated. These points 230 can then be converted into, and recorded as, digital three-dimensional geometrical locations within a coordinate system associated with the treatment device coordinate system, room coordinate system, and/or another useful coordinate system.

In an alternative embodiment of the invention, a more complete and/or automatic representation of a surface region of a patient may be obtained. An example embodiment using a more thorough surface measurement system is illustrated in FIG. 2B, in which a measurement system 240 such as, but not limited to, a camera, projector or laser scanning device can be used to acquire surface information over a greater number of locations, and store this information digitally as geometrical information within a defined coordinate system, and/or as pictorial information.

The embodiments described above for FIGS. 2A and 2B can be used to acquire patient surface information calibrated to a coordinate system 250 related to a position on, or within, the patient. The patient coordinate system 250 can then be related to a coordinate system associated with the treatment room, a radiation delivery device, or both, using one or more transformations obtained using various known calibration techniques. Alternatively, the surface measurements can be stored directly within a coordinate system based on the treatment room and/or the device without the need for transforming from one coordinate system to another. For example, a marker tool 210 can be calibrated to the coordinate system 250 at known points along the coordinate system 250 and can then use these points to define a transformation between the tracker's position in three-dimensional space and the coordinate system associated with a treatment-delivery device.

In an alternative embodiment of the invention, a projector/camera system or laser scanner is calibrated to the coordinate system 250 by identifying known points along the coordinate system 250 in images acquired previously with the device, and relating the images of these points to their known positions in a second, room-based, coordinate system.

In one embodiment, a wall or ceiling-mounted optical camera can be used to calibrate images taken using a hand-held ultrasound imaging probe to a three-dimensional reference coordinate system defined in a radiation-treatment room. However, it is to be understood that the present invention may be applied to detecting calibration errors for virtually any tracking device, such as, but not limited to, optical, magnetic, or mechanical devices, in essentially any environment.

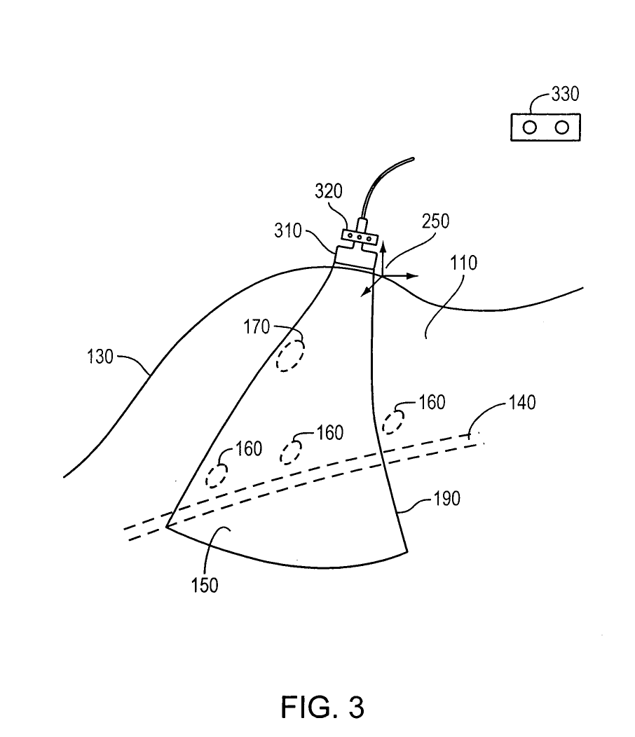

In addition to the acquisition of external surface information, acquisition of internal information regarding the location, size, and/or shape of structures within the region of interest of a patient is also obtained. For example, one important feature of internal patient information for the delivery of radiation therapy to the breast is the lung/chest wall or pleura interface 140, although other features such as the tumor bed, heart, or nodes may typically also be of interest. As shown in FIG. 3, an ultrasound device 310 may be used in the treatment room to acquire images showing these various anatomical features of a patient as they appear at the time of treatment delivery. Ultrasound is a generally preferred method of imaging internal anatomical features as it is less expensive than other in-room imaging devices (e.g., cone-beam CT) and does not emit ionizing radiation. However, other means of imaging internal anatomical features may also be utilized in alternative embodiments of the invention.

In one embodiment, the ultrasound device 310 includes a hand-held probe with attached sensors 320 so that the position and the orientation of the probe can be tracked by an optical tracking device 330 using the same coordinate system 250 associated with the external surface information. In one embodiment the optical tracking device 330 can be the same device as used for the tracking of the external tracking system, while in another embodiment the tracking device may be associated only with the ultrasound device 310, or other internal measurement device, and be associated with a distinct (but related) coordinate system. In an alternative embodiment, the position and/or orientation of the probe ultrasound device 310, or other internal measurement device, can be obtained by another means, such as, but not limited to, a magnetic tracker system or a mechanical arm.

Using the ultrasound device 310 or other internal measurement device, a full three-dimensional ultrasound image can be constructed (from individual two-dimensional images, for example) in the coordinate system 250 which can subsequently be viewed in any arbitrary plane. This may be achieved, in one embodiment, by creating a three-dimensional image by combining a plurality of two-dimensional images (or “slices”), with each two-dimensional slice offset from the others, to produce a data set spanning a three-dimensional volume. The pleura-lung interface 140, and other organs, can then be identified by the user. In an alternative embodiment, the relevant internal features of the patient can be identified automatically using a conventional segmentation algorithm.

In a further alternative embodiment, a series of one or more two-dimensional frames can be acquired, with their position and orientation determined using one or more of the methods outlined above, to obtain a smaller subset of points on the lung/chest wall interface. In another alternative embodiment, a three-dimensional ultrasound device is used to capture a complete three-dimensional image. The ultrasound device can be calibrated to the same coordinate system 250 associated with the device used to identify and/or capture external surface information, which itself can be related to coordinates associated with the radiotherapy treatment room and/or the radiation-treatment device. This can be accomplished by scanning an ultrasound “phantom” with embedded structures at known positions within the coordinate system, identifying the structures in the images and mathematically relating the known positions to the positions in the images. Such methods are described in pending U.S. patent application Ser. No. 11/184,745 entitled “Calibrating Imaging Devices,” the entire disclosure of which is incorporated herein by reference in its entirety.

Using the techniques described above, the differences in external surface and internal anatomy encountered prior to treatment delivery can be considered and accounted for during the treatment phase. As such, differences between the treatment plan and the actual treatment delivered to the location of interest can be minimized.

In one exemplary embodiment, a coordinate system may be associated with multiple aspects of the treatment, with an appropriate transformation between each coordinate system allowing for a full representation of the patient's external and internal anatomy with respect to the treatment room and/or treatment device. For example, external measurements may be taken with respect to a coordinate system associated with an optical tracking device, while internal measurements may be taken with respect to a coordinate system associated with the ultrasound device used to measure the internal anatomical features of the patient. So long as the different coordinate systems are related by a known transformation, data from one coordinate system can be accurately mapped into the other.

By using an optical tracking device to track the position and orientation of the ultrasound instrument, the internal anatomical measurements can be transformed into data in a coordinate system associated with this tracking device. It should be noted that the optical tracking device for the ultrasound instrument may be the same optical tracking device associated with the external measurements, or may be a separate, distinct optical tracking device. The data in the coordinate system associated with the one or more optical tracking devices can then be subjected to a simple transformation to provide both external and internal anatomical position data in a coordinate system associated with the treatment room or treatment device. This facilitates simple comparison with prior data and quick adjustment of the treatment device, and/or patient position, to compensate for any differences in the patient anatomical data from the treatment-plan measurements to the most current measurements.

In some prior-art methods of treating an internal structure, such as a cancerous lesion in a breast, marks placed on the external surface (e.g., along the contour of the breast) are used for determining beam placement and angles for breast patients. To accurately-position the beam, one required component of the calculations is the determination of the chest wall plane. However, the determination of the chest wall plane using marks on the external surface does not account for actual changes in the position of the chest wall/lung interface relative to the patient contour, and as such can result in misalignment of the beam during treatment. Using ultrasound data, as described herein, a chest wall plane can be identified and used to calculate the correct treatment parameters instead of (or in addition to) relying exclusively on the external markings.

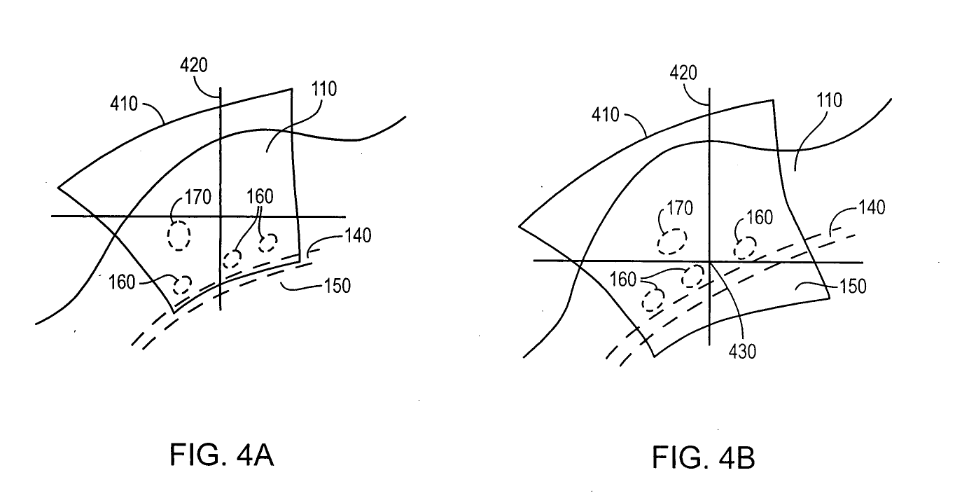

An exemplary configuration for a radiation treatment prior to correction of the beam position can be seen in FIG. 4A. Here, a first radiation beam 410 is shown relative to the coordinate system 420, lesion 170, and other anatomical features of the patient P, such as the pleura 140, lung 150, and ribs 160. In FIG. 4A, despite the coordinate system 420 being correctly aligned with respect to the external surface features of the region of interest, in this case the patient's breast, changes in the position of the chest wall/lung interface, lesion, and other internal features of the patient relative to the patient contour are not accounted for. As a result, the coordinate system 420 is not centered at the position defined during the treatment-planning stage, resulting in a less-than-optimal treatment delivery. This may result in a smaller than required radiation dose reaching the lesion 170, while portions of the surrounding non-cancerous tissue may be exposed to higher levels of radiation than is expected and/or safe.

By measuring both the external and internal features of the patient at the time of treatment, a shifting of the chest wall relative to the patient's breast (and, therefore, to the external markings on the breast) may be accounted for. As a result, the isocenter (or any combination of other treatment parameters) of the radiation beam 410 can be adjusted in accordance therewith, thus resulting in the beam 410 being properly aligned with respect to the lesion 170. An example of a correctly aligned coordinate system 420 and radiation beam 410 can be seen in FIG. 4B. In this embodiment, the isocenter 430 of the coordinate system 420 is located below the lesion 170. In other contexts, the isocenter may be positioned at the center of the lesion 170, or at a different location around the lesion 170, depending upon the treatment required by the treatment plan. In general, parameters such as, but not limited to, lesion size and structure, number of lesions, and or structure and location of surrounding tissue and organs, may be considered during the treatment planning phase in order to determine the optimum location of the isocenter in a particular case.

In one exemplary embodiment, the measured external information and the measured internal anatomical information are used to determine whether different parameters of the treatment system require adjustment prior to treatment. For example, the external measurements may be used to determine whether one or more beam parameters requires adjustment. These beam parameters may include, but are not limited to, the angle of the beam collimator, the strength of the beam, the focal length of the beam, or any other appropriate parameter effecting the radiotherapy beam being delivered. Upon determining that the external geometry of the breast has changed from that measured during treatment planning, one or more of these beam parameters is adjusted either automatically, by a control algorithm associated with the control system, or manually by the medical practitioner using the apparatus. Changing one or more of these parameters can change the angle of entry of the beam, change the isocenter of the beam, and/or change the length of time the beam is on, to compensate for the changed external geometry and ensure that the correct radiotherapy dose is delivered.

In addition, the internal anatomical measurements may be compared to the previously measured internal anatomy to determine whether the position of the patient with respect to the radiotherapy beam system should be adjusted. For example, if it is determined that the lesion is now further from the skin than at the time of the treatment planning measurements, the patient may be moved closer to the source of the radiation beam to compensate. This adjustment of the patient's position may be carried out by adjusting one or more adjustable degrees of freedom of the patient support device. This adjustment can again be carried out automatically in response to an instruction from a control algorithm, or be carried out manually be the medical practitioner. The adjustment of the patient may include, but is not limited to, raising or lowering the patient, moving her in the plane perpendicular to the beam axis, or changing the angle of the patient with respect to the delivery device.

Both the external and internal anatomical measurements may be used to determine whether a change to either one or more beam parameters, and/or the patient position, is required. For example, although changes in the external measurements usually imply the need for changes in one or more of the beam parameters, this may be so only within a predetermined range, beyond which resort to changes in patient position—with or without changes in the beam parameter(s) as well—are called for. Analogously, large-scale changes in the internal measurements may call for alteration of one or more beam parameters in lieu of or in addition to changes in patient positioning. Finally, the external and/or the internal anatomical information may be used to determine whether a full recalculation of the treatment plan is required, and be used to prepare this updated treatment plan.

In one embodiment, a threshold degree of difference from the treatment plan data to the presently measured data is set, beyond which a full recalculation to the treatment plan is required. In this embodiment, measurements of both the external and internal anatomical geometry of the patient are taken prior to a treatment session. These results are then compared to the anatomical data taken at the time of creation of the treatment plan. If there is no therapeutically meaningful difference between the present data and the treatment plan data, then treatment can commence immediately in accordance with the treatment plan. However, if changes to the external and/or the internal anatomical geometry are observed relative to the original treatment plan, these may be compensated for by adjusting one or more parameters associated with the system as described above.

Here, it can first be determined whether the differences in the external and/or internal data are lower than a predetermined threshold amount. If the differences are below these thresholds, the external data may be used to determine an appropriate adjustment of one or more beam parameters, while the internal data may be used to determine an appropriate adjustment of the patient position, as described above. However, if the difference between the present measurements and the stored treatment plan data, for either the external or internal data, exceeds the set threshold, a more involved adjustment and/or recalculation may be required. This may involve adjusting the beam parameter(s) and/or patient position. Alternatively, if all threshold values are exceeded, a partial or complete recalculation of the treatment plan may be required.

In one embodiment, the system provides a signal to the user indicating that a threshold difference between the present anatomical data and stored treatment plan data has been exceeded. This signal may include, but is not limited to, any appropriate visual and/or acoustical signal. Alternatively, exceeding a threshold value may result in the treatment system automatically recalculating the treatment plan and adjusting one or more system parameters in accordance with the new plan. In a further alternative embodiment, a plurality of threshold values may be set, with different system responses depending upon the specific threshold exceeded.

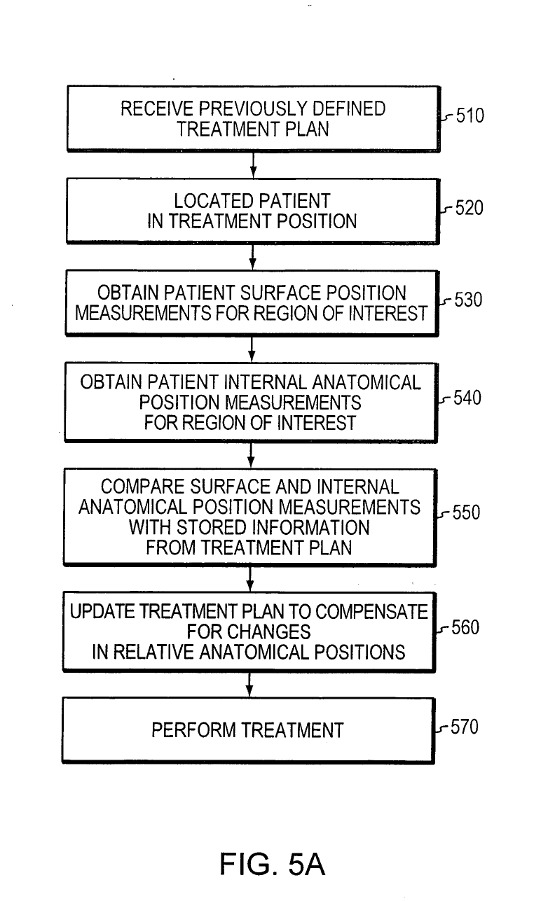

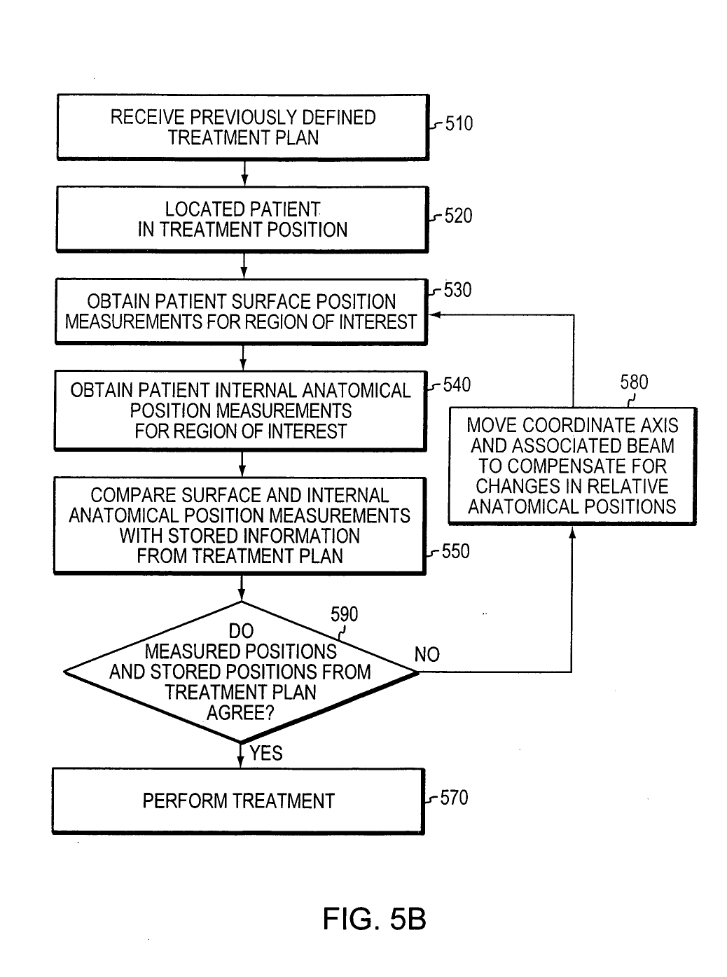

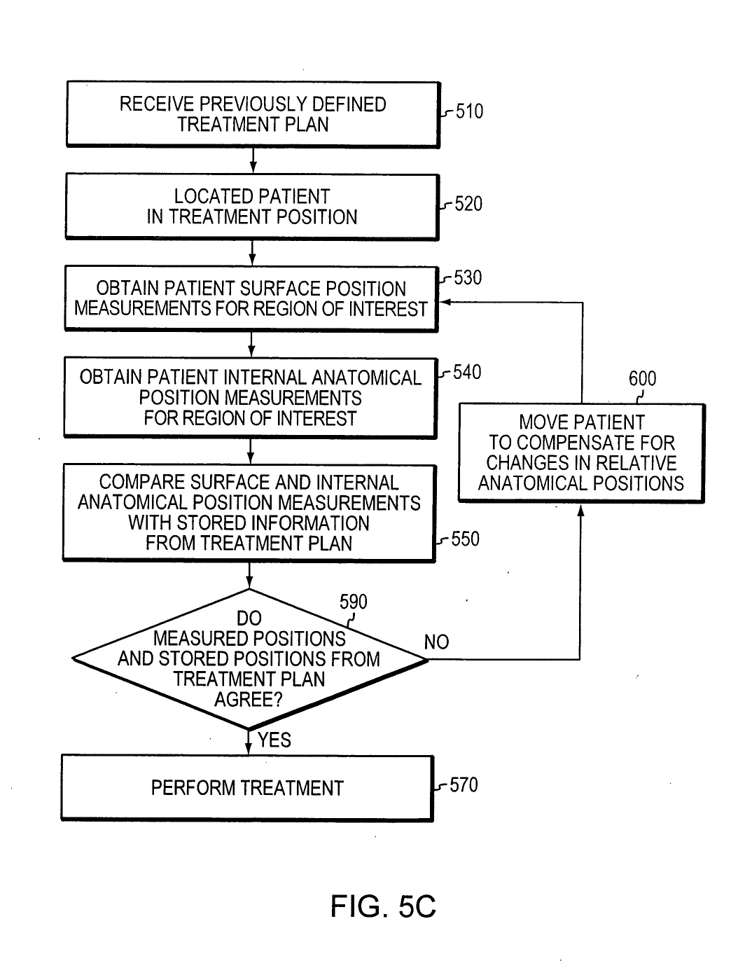

Illustrative embodiments of methods for carrying out the invention can be seen in FIGS. 5A-5C. More specifically, the method illustrated in FIG. 5A involves receiving a previously defined treatment plan (step 510). This may include one or more of inputting and/or downloading stored digital information into a control/measurement system, inputting one or more parameters defining the treatment into a control/measurement system for the therapy delivering equipment, and/or providing a user with information necessary to carry out the method and treatment procedure, such as, but not limited to, providing pictorial, graphical, and numerical data associated with the patient and required treatment.

The patient may then be located on a treatment table in a required treatment position (step 520), which may be the same position as in the investigation carried out to produce the treatment plan. Once correctly positioned, surface position measurements (step 530) and internal anatomical position measurements (step 540) may be obtained. The results of these measurements can then be compared with the information stored in the treatment plan (step 550). These results may be compared manually by a user and/or automatically by the control/measurement system for the measurement and treatment system. If the measured position measurements do not conform to those stored in the treatment plan, the treatment plan may be updated (step 560) to compensate for these changes in order to ensure that the required treatment is still delivered to the correct location. This updating of the treatment plan may involve changing the power of the radiation beam, the length of delivery, or variation of some other delivery parameter.

Alternatively, the updating of the treatment plan may involve moving the beam-delivery device to locate the coordinate axis for the beam at the correct location and orientation (step 580), as shown in FIG. 5B. Once this movement has been performed, the surface and internal measurements may be obtained again to ensure that the correct position and orientation of the coordinate system with respect to the patient has been achieved. If the measured and stored positions do agree (step 590), the treatment may be performed (step 570) as required by the treatment plan. In an alternative embodiment the surface and internal measurements are not repeated, but rather the treatment commences without further steps upon the repositioning of the coordinate axis. In a further alternative embodiment illustrated in FIG. 5C, the patient, rather than the coordinate axis and beam, may be repositioned (step 600) to ensure that the radiation is delivered to the correct location.

Using such techniques, or a combination thereof, any adjustments made to the radiotherapy beams prior to each treatment session can be based on both surface information and ultrasound-based internal anatomy, where the images are referenced in the same or related coordinate systems. As a result, the required treatment may be accurately delivered to the correct location, and at the correct angle, regardless of the time between treatments and even the location of the treatment.

In an alternative embodiment, an automated computer planning system capable of calculating dosages and other treatment parameters generates a new treatment plan prior to each treatment session, taking dose calculations and the newly determined patient anatomy positioning into account. Based on patient surface and lung information, an optimization routine finds the best beam shapes and dosages to deliver a uniform dose to the breast while minimizing lung dose, or, in some cases, to minimize the difference in doses between the treatment plan and the dose calculated on the current treatment anatomy.

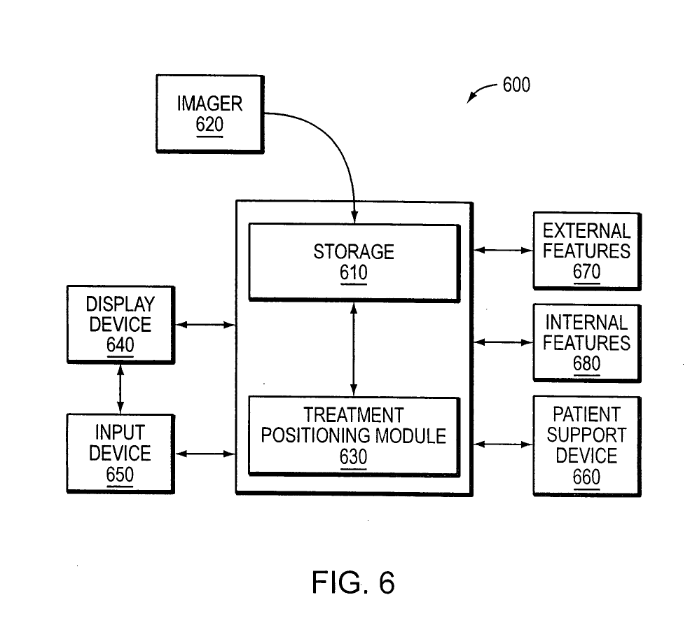

Referring to FIG. 6, one embodiment of a system 600 for performing the techniques described above includes a storage device 610 that is configured to receive image data from an imaging device 620 (such as a hand-held ultrasound device) via a cord or wire, or in some embodiments via wireless communications. In one embodiment, the storage device 610 can also receive data from a device configured to map a portion of the external surface of a patient, such as a pointer tool, camera, or laser scanner. In an alternative embodiment, a receiver can be used to receive and store data from an external mapping device.

The system also includes a treatment-positioning module 630 that, based on the image data, uses the techniques described above to compare the measured internal anatomy data and/or external surface data with stored information of the treatment area from a treatment plan. In some embodiments, the system also includes a display 640 and an associated user interface (not shown) that allows a user to view and manipulate the stored and measured ultrasound images and/or surface position images/data. The display 640 and user interface can be provided as one integral unit or separate units (as shown) and may also include one or more user input devices 650 such as a keyboard and/or mouse. The display 640 can be passive (e.g., a “dumb” CRT or LCD screen) or in some cases interactive, facilitating direct user interaction with the images and models through touch-screens (using, for example, the physician's finger as an input device) and/or various other input devices such as a stylus, light pen, or pointer. The display 640 and input devices 650 may be proximate to or remote from the storage device 610 and/or treatment positioning module 630, thus allowing users to receive, view, and manipulate images in remote locations using, for example, wireless devices, handheld personal data assistants, notebook computers, among others.

The system can further include a patient support device 660 for adjusting the position of the patient with respect to a treatment delivery device, such that the treatment is delivered to the correct location and at the correct angle, as required by the patient treatment plan. This patient support device 660 may, in one embodiment, include movable structure for supporting at least a portion of a patient, such that the position and orientation of the patient may be moved in response to instructions from the treatment positioning module 630, or through direct user input. In one embodiment of the invention, hydraulic and/or electromagnetic devices can be installed in the patient support device 660 to provide means for varying the location and orientation of the patient with respect to a given coordinate system.

In various embodiments the storage device 610 and/or treatment positioning module 630 may be provided as either software, hardware, or some combination thereof. For example, the system may be implemented on one or more server-class computers, such as a PC having a CPU board containing one or more processors such as the Pentium or Celeron family of processors manufactured by Intel Corporation of Santa Clara, Calif., the 680×0 and POWER PC family of processors manufactured by Motorola Corporation of Schaumburg, Ill., and/or the ATHLON line of processors manufactured by Advanced Micro Devices, Inc., of Sunnyvale, Calif. The processor may also include a main memory unit for storing programs and/or data relating to the methods described above. The memory may include random access memory (RAM), read only memory (ROM), and/or FLASH memory residing on commonly available hardware such as one or more application specific integrated circuits (ASIC), field programmable gate arrays (FPGA), electrically erasable programmable read-only memories (EEPROM), programmable read-only memories (PROM), programmable logic devices (PLD), or read-only memory devices (ROM). In some embodiments, the programs may be provided using external RAM and/or ROM such as optical disks, magnetic disks, as well as other commonly storage devices.

For embodiments in which the invention is provided as a software program, the program may be written in any one of a number of high level languages such as FORTRAN, PASCAL, JAVA, C, C++, C^{#}, LISP, PERL, BASIC or any suitable programming language. Additionally, the software can be implemented in an assembly language and/or machine language directed to the microprocessor resident on a target device.

The invention may be embodied in other specific forms without departing form the spirit or essential characteristics thereof. The foregoing embodiments, therefore, are to be considered in all respects illustrative rather than limiting the invention described herein. Scope of the invention is thus indicated by the appended claims, rather than by the foregoing description, and all changes that come within the meaning and range of equivalency of the claims are intended to be embraced therein.

What is claimed is:

1. A method for determining an adjustment to be applied to a radiation treatment plan, the method comprising the steps of:
obtaining a radiation treatment plan comprising a plurality of treatment parameters including at least the position of a patient and external and internal anatomical features of the patient;
obtaining a visual representation of at least one external feature of the patient in reference to a first reference coordinate system;
at substantially the same time as the external-feature visual representation is obtained, obtaining a visual representation of at least one internal anatomical feature of the patient in reference to a second reference coordinate system; and
determining the adjustment based on the visual representations.

2. The method of claim 1 further comprising adjusting one or more of the treatment parameters to compensate for changes in the position of the patient relative to a radiation treatment device based on the visual representations.

3. The method of claim 2, wherein the visual representation of at least one external feature is used to determine an adjustment required in at least one radiotherapy beam parameter.

4. The method of claim 2, wherein the visual representation of at least one internal anatomical feature is used to determine an adjustment required in at least one patient position parameter.

5. The method of claim 1, further comprising establishing a threshold value below which an adjustment of one or more treatment parameters is not required.

6. The method of claim 1, further comprising establishing a threshold value above which a full recalculation of the treatment plan is required.

7. The method of claim 1 wherein the at least one external feature of the patient comprises a naturally occurring feature.

8. The method of claim 1 wherein the at least one external feature of the patient comprises an artificial mark place on the patient.

9. The method of claim 1 wherein the at least one external feature of the patient comprises surface elements representative of the patient's skin.

10. The method of claim 1 wherein the radiation treatment plan comprises one or more doses of radiation to be delivered to the patient's breast.

11. The method of claim 1 wherein the visual representation of the at least one external feature is obtained using a camera.

12. The method of claim 1 wherein the visual representation of the at least one external feature is obtained using a tracking tool.

13. The method of claim 1 wherein the visual representation of the at least one external feature is obtained using a laser scanning device.

14. The method of claim 1 wherein the visual representation of the at least one internal feature is obtained using an ultrasound imaging device.

15. The method of claim 10 wherein the ultrasound imaging device produces three-dimensional ultrasound images.

16. The method of claim 10 wherein the ultrasound imaging device produces a two-dimensional image and further comprising mapping the two-dimensional image into three-dimensional space.

17. The method of claim 1 wherein the visual representation of the patient's internal feature is obtained using an x-ray imaging device.

18. The method of claim 1 wherein the treatment parameters comprise one or more of an isocenter, a couch angle, a beam angle, a couch position, a radiation dosage, a wedge angle, a collimator size, a collimator shape, and a collimator angle.

19. The method of claim 1 wherein the first reference coordinate system and the second reference coordinate system are the same reference coordinate system.

20. The method of claim 1 wherein the first reference coordinate system and the second reference coordinate system are related by a transformation.

21. A system for determining an adjustment to be applied to a radiation treatment plan, the system comprising:
storage for storing:
a plurality of parameters including at least the position of a patient and external and internal anatomical features of the patient;
a representation of at least one external feature of the patient in reference to a first reference coordinate system; and
a representation of at least one internal anatomical feature of the patient in reference to a second reference coordinate system, and
a positioning module in communication with the storage for adjusting, based on the visual representations, one or more of the parameters to compensate for changes in the position of the patient with respect to the at least one patient internal feature.

22. The system of claim 21 further wherein the positioning module further provides instructions to a patient support device for adjusting the position of the patient.

23. The system of claim 21 further comprising a tracking device for tracking the at least one patient external feature.

24. The system of claim 21 further comprising a camera for obtaining the visual representation of the at least one patient external feature.

25. The system of claim 21 further comprising a tracking device for tracking the at least one patient internal anatomical feature.

26. The system of claim 21 further comprising an ultrasound imaging device to obtain the visual representation of the at least one patient internal anatomical feature.

27. The system of claim 24 further including an optical tracking device for monitoring the location of the ultrasound imaging device with respect to the second reference coordinate system.

28. The system of claim 21 wherein the treatment parameters comprise one or more of an isocenter, a couch angle, a couch position, a beam angle, a radiation dosage, a wedge angle, a collimator size, a collimator shape, and a collimator angle.

29. The system of claim 21 wherein the first reference coordinate system and the second reference coordinate system are the same reference coordinate system.

30. The system of claim 21 wherein the first reference coordinate system and the second reference coordinate system are related by a transformation.

31. A method for determining a radiation treatment plan, the method comprising the steps of:
obtaining a visual representation of at least one external feature of a patient with respect to a reference coordinate system;
at substantially the same time as the external-feature visual representation is obtained, obtaining a visual representation of at least one internal anatomical feature of the patient with respect to the reference coordinate system; and
determining a radiation treatment plan comprising a plurality of treatment parameters in the reference coordinate system based on the position of the patient relative to the external and internal anatomical features in the reference coordinate system.

32. The method of claim 31 wherein the radiation treatment plan is determined substantially contemporaneously with obtaining the visual representation of at least one internal anatomical feature of the patient.

33. The method of claim 32 wherein the radiation treatment plan is determined substantially contemporaneously with delivery of the radiation treatment.
